(19) United States
(12) Patent Application Publication
(10) Pub. No.: US 2024/0409879 A1
(43) Pub. Date: Dec. 12, 2024

(54) SYSTEMS AND METHODS FOR CULTIVATING ALGAE USING CARBONIC ANHYDRASE

(71) Applicant: Global Algae Technology, LLC, San Diego, CA (US)

(72) Inventors: David A. Hazlebeck, El Cajon, CA (US); Rodney Corpuz, Lihue, HI (US); William Rickman, Lebanon, TN (US)

(21) Appl. No.: 18/740,263

(22) Filed: Jun. 11, 2024

Related U.S. Application Data
(60) Provisional application No. 63/472,497, filed on Jun. 12, 2023.

(51) Int. Cl. C12N 1/12; C12M 1/00; C12N 1/20; C12N 9/88

(52) U.S. Cl. CPC C12N 1/12; C12M 21/02; C12M 23/34; C12M 23/40; C12M 29/18; C12M 47/10; C12N 1/20; C12N 9/88; C12Y 402/01001

(57) ABSTRACT

Described herein are systems and methods for supplying an algae cultivation fluid with carbon by incorporating microorganisms that express carbonic anhydrase. The systems and methods further include capturing carbon dioxide directly from the atmosphere for absorption by the carbonic anhydrase.

### CROSS REFERENCE TO RELATED APPLICATIONS

This application claims the benefit of U.S. Provisional Application No. 63/472,497, filed Jun. 12, 2023, the content of which is hereby incorporated by reference in its entirety.

### STATEMENT REGARDING FEDERALLY SPONSORED RESEARCH

This invention was made with government support under Grant Nos. DE-EE0008639, subcontract JCVI-19-011, and DE-EE0009758 awarded by the U.S. Department of Energy. The government has certain rights in the invention.

### BACKGROUND

Algae cultivation utilizes a supply of nutrients and photosynthesis to fix CO₂ for growth. Typically, high intensity cultivation in an algae farm utilizes the addition of CO₂ in some form to support a high productivity. In some cases, pure CO₂ is bubbled into raceways to support high rates of photosynthesis. This approach enables locating algae farms almost anywhere, but the cost of buying the CO₂ is high, typically over $100 per ton in 2020 dollars. Utilizing a moderately concentrated CO₂ source, e.g. 1% to 20% CO₂ by volume, such as combustion flue gas is less expensive, but its use is limited by the need to locate the algae farm near the source. Methods for capturing CO₂ directly from the atmosphere using algae farms have been developed. Not only do these direct air capture methods significantly reduce farming costs and farm location limitations, but they are also a source of high volumes of excess CO₂. The CO₂ may be sequestered to produce polymer products, such as consumer plastics, from oil produced from the algae. The CO₂ may also be released on use of the algae oil for feed, food, and jet fuel. These processes may lower greenhouse gas emissions and address deforestation. Therefore, methods for maximizing CO₂ absorption during algae cultivation are desired.

It has been previously shown that carbonic anhydrase increases CO₂ absorption rates when added to an algae culture. However, producing, isolating, and transporting carbonic anhydrase to an algae culture is too expensive for practical application. Therefore, more cost-efficient means for providing carbonic anhydrase to an algae culture are desired.

### SUMMARY OF THE INVENTION

An objective of the invention is to utilize microorganism-produced carbonic anhydrase to increase the absorption rate of carbon dioxide without the need to separate and purify the carbonic anhydrase.

Disclosed herein is an algae cultivation system including: an algae cultivation unit (1) configured to produce an algae slurry in a cultivation fluid; a microorganism cultivation unit (5) configured to produce a microorganism slurry in a growth media under conditions in which one or more microorganisms produce carbonic anhydrase; and a growth media conduit (8) configured to transfer at least a portion of the microorganism slurry from the microorganism cultivation unit (5) to the algae cultivation unit (1).

Also disclosed herein is an algae cultivation system including: an algae cultivation unit (1) configured to produce an algae slurry in a cultivation fluid; a microorganism cultivation unit (5) configured to produce a microorganism slurry in a growth media under conditions in which one or more microorganisms produces and excretes carbonic anhydrase into the growth media; a microorganism harvesting system (9) configured to separate the microorganism from the growth media and generate a filtered media; a growth media conduit (8) configured to transfer the microorganism slurry from the microorganism cultivation unit (5) to the microorganism harvesting system (9); and a filtered media conduit (10) configured to transfer at least a portion of the filtered media from the microorganism harvesting system (9) to the algae cultivation unit (1).

Further disclosed herein is an algae cultivation system including: an algae cultivation unit (1) configured to produce an algae slurry in a cultivation fluid; a microorganism cultivation unit (5) configured to produce a microorganism slurry in a growth media under conditions in which one or more microorganisms produces carbonic anhydrase; a lysis system (12) configured to lyse the microorganism and release carbonic anhydrase into the growth media; a growth media conduit (8) configured to transfer the microorganism slurry from the microorganism cultivation unit (5) to the lysis system (12); a lysed microorganism slurry conduit (13) configured to transfer at least a portion of the lysed microorganism from the lysis system (12) to the algae cultivation unit (1).

In another instance, this disclosure provides for an algae cultivation system including: an algae cultivation unit (1) configured to produce an algae slurry in a cultivation fluid; a harvest system (3) configured to produce a concentrated algae product from the algae slurry; a slurry conduit (2) configured to transfer the algae slurry from the algae cultivation unit (1) to the harvest system (3); a microorganism cultivation unit (5) configured to produce a microorganism slurry in a growth media under conditions in which one or more microorganisms produces carbonic anhydrase; a lysis system (12) configured to lyse the microorganism and release carbonic anhydrase into the growth media; a growth media conduit (8) configured to transfer the microorganism slurry from the microorganism cultivation unit (5) to the lysis system (12); a filtration system (14) configured to separate the microorganism suspended cell debris from the growth media and produce a filtered lysis media; a lysed microorganism slurry conduit (13) configured to transfer at least a portion of the lysed microorganism slurry from the lysis system (12) to the filtration system (14); and a filtered lysis media conduit (15) configured to transfer at least a portion of the filtered lysis media from the filtration system (14) to the algae cultivation unit (1).

Disclosed herein is also a method of cultivating an algae culture in a cultivation fluid including: (i) cultivating a microorganism that produces carbonic anhydrase in a growth media under conditions in which the microorganism produces and excretes carbonic anhydrase into the growth media; and (ii) providing the at least a portion of growth media to the algae culture.

Provided herein is a method of cultivating an algae culture in a cultivation fluid including: (i) cultivating a microorganism that produces carbonic anhydrase in a growth media under conditions in which the microorganism produces the carbonic anhydrase; (ii) lysing the microorganism in the growth media; and (iii) providing at least a portion of the growth media to the algae culture.

These and other advantages and features of the invention will become more apparent from the following detailed description of the preferred embodiments of the invention when viewed in conjunction with the accompanying drawings.

### BRIEF DESCRIPTION OF THE DRAWINGS

FIG. 1 is a schematic illustration of an algae cultivation system in accordance with some embodiments of the present disclosure.

FIG. 2 is a schematic illustration of an algae cultivation system in accordance with some embodiments of the present disclosure.

FIG. 3 is a schematic illustration of an algae cultivation system in accordance with some embodiments of the present disclosure.

FIG. 4 is a schematic illustration of an algae cultivation system in accordance with some embodiments of the present disclosure.

FIG. 5 is a schematic illustration of an algae cultivation system in accordance with some embodiments of the present disclosure.

FIG. 6 shows a plot of pH versus time of samples prepared from a 0.02 M algae pond.

FIG. 7 shows a plot of pH versus time of samples prepared from a 0.05 M algae pond.

FIG. 8 shows a plot of pH versus time of samples prepared from a 0.1 M algae pond.

FIG. 9 shows a plot of pH versus time of samples prepared from a 0.3 M algae pond.

FIG. 10 shows a plot of pH versus time of samples prepared from a 0.3 M algae pond, without recycle.

FIG. 11 shows a chart showing the slope ratio of plots provided in FIG. 6 through FIG. 10, where the slope is the averaged rate of pH decrease, and the slope ratio is the sample slope divided by the slope of an inorganic media sample at the same pH, molarity, and temperature.

### DETAILED DESCRIPTION

Disclosed herein are systems and methods for algae cultivation using direct air capture with carbonic anhydrase. Carbonic anhydrase is a zinc metalloenzyme that catalyzes the interconversion of carbon dioxide (CO₂) and water (H₂O) into carbonic acid (H₂CO₃), protons (H⁺), and bicarbonate ions (HCO⁻₃). The enzyme was first discovered in red blood cells but has since been found in most organisms including animals, plants, archaebacteria, and eubacteria. Carbonic anhydrase (CA) is important in many physiological functions that involve carboxylation or decarboxylation reactions, including both photosynthesis and respiration. In addition, CA also participates in the transport of inorganic carbon to actively photosynthesizing cells or away from actively respiring cells.

Known carbonic anhydrases can be grouped into at least eight independent families. The three primary families are: α-CA, β-CA, and γ-CA. These three families have no primary sequence similarities and may have evolved independently. The complete distribution of these CAs is uncertain. Plants appear to have all three types of CAs. Cyanobacteria have both α-CA and β-CA and the CcmM protein, a protein which functions as a scaffold protein for the assembly of β-carboxysome and that bears strong similarity to γ-CA. Examples of α-CA and β-CA are known in algae. CA have been found in the mitochondria, the chloroplast thylakoid, the cytoplasm and the periplasmic space of algae.

Although the primary sequences of these CA families are different, all three types of carbonic anhydrases are Zn²⁺ metalloenzymes and all appear to share a similar catalytic mechanism. In all cases it appears that a Zn—OH⁻ attacks a CO₂ molecule residing in a hydrophobic pocket, generating a Zn-bound HCO3⁻ (Equation 1). The bicarbonate bound to the zinc is then replaced by a water molecule, releasing HCO₃⁻. HCO₃⁻ in solution can gain a H⁺ to form H₂CO₃ or can lose an additional H⁺ to form CO₃⁻². The overall relationship between the three forms of dissolved inorganic carbon is given as:

CO₂+H₂O↔HCO₃⁻+H⁺↔CO₃²⁻+2H⁺ (1)

The equilibrium between the inorganic carbon forms is pH dependent. At normal intracellular ionic strength, when the pH level is below the first dissociation constant (pK₁˜6.4), CO₂ predominates; at pH between 6.4 and about 10.3 (pK₂) HCO₃⁻ predominates, whereas above pH of 10.3, CO₃²⁻ predominates. The uncatalysed hydration-dehydration reactions are slow, whereas the dissociation reactions are considered instantaneous. Carbonic anhydrase greatly accelerates the hydration of dissolved CO₂ in solution thereby increasing the rate at which forms of inorganic carbon interconvert in solution.

As noted above, high levels of algae biomass productivity (g/m²d) utilize supplementation of gaseous nutrients, such as carbon dioxide, for cultivation. Typically, concentrated carbon dioxide or flue gas are added to algae cultures as a source of carbon dioxide. However, this approach increases operating costs of cultivation farms, and in the case of flue gas utilization, can limit where a farm may be located. Adding carbonic anhydrase to an algae culture increases the adsorption rate of carbon dioxide, however commercially available sources of carbonic anhydrase are prohibitively expensive for practical application at scale. The present disclosure addresses the aforementioned drawbacks by providing systems and methods for supplying an algae cultivation fluid with carbon dioxide directly from air (e.g., gases within the atmosphere).

The present disclosure provides improved systems and methods for algae cultivation using carbonic anhydrase production. In some embodiments, the systems and methods provided herein are performed without applying, supplementing, or treating an algae cultivation fluid with concentrated carbon dioxide and/or flue gas. Examples of direct air capture algae cultivation systems and methods are described in U.S. Patent Publication No. 2021/0386029A1, which is incorporated by reference, in its entirety and for all purposes, herein.

As used herein, the terms “air” and “atmosphere” may refer to gases surrounding the earth, which may vary regionally, and are a function of various factors, such as temperature and pressure. As one example, the terms “air” and “atmosphere” may refer to a gaseous composition composed, in a dry volume percentage (vol %), of about 78 vol % nitrogen, about 20.9 vol % oxygen, about 0.9 vol % argon, about 0.04 vol % carbon dioxide, and other elements and compounds such as helium, methane, krypton, hydrogen, nitrous oxide, xenon, ozone, carbon monoxide, sulfur dioxide, nitrogen dioxide, and ammonia. As used herein, the term “atmospheric carbon dioxide” may refer to carbon dioxide derived from air, and the term “atmospheric nitrogen” refers to nitrogen derived from air.

### Systems

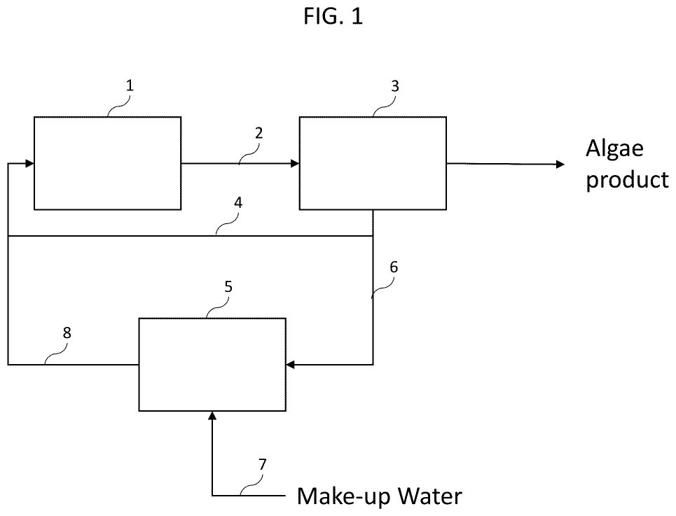

Referring to FIG. 1, one embodiment of the algae cultivation system includes an algae cultivation unit (1) configured to produce an algae slurry in a cultivation fluid, a harvest system (3) configured to produce a concentrated algae product from the algae slurry and a recycled media, a slurry conduit (2) configured to transfer the algae slurry from the algae cultivation unit (1) to the harvest system (3), a microorganism cultivation unit (5) configured to produce a microorganism slurry in a growth media under conditions in which one or more microorganisms produces and/or excretes carbonic anhydrase into the growth media, a growth media conduit (8) configured to transfer at least a portion of the algae culture and the growth media from the microorganism cultivation unit (5) to the algae cultivation unit (1), a main recycled media conduit/a first recycled cultivation fluid conduit (4) configured to transfer at least a portion of the growth media from the harvest system (3) to the algae cultivation unit (1), and a secondary recycled media conduit (6) to optionally transport a portion of the recycled media to the microorganism cultivation unit (5).

The system may further comprise a make-up water conduit (7), configured to transfer water to the microorganism cultivation unit (5). In embodiments, the water may be provided from outside of the system. The water may include make-up water, replacing water that may have been lost, e.g., as a result of evaporation from the cultivation unit and/or water removed from the further processed algae product output of the harvest system (3).

In embodiments, the algae cultivation unit (1) is configured to capture carbon dioxide directly from air. As noted above, suitable methods and systems for the capture of carbon dioxide directly from air are provided in U.S. Patent Publication No. 2021/0386029A1, which is incorporated by reference, in its entirety, herein. For example, one method of capturing carbon dioxide from air may include applying bore waves through the cultivation fluid at a bore wave frequency sufficient to disrupt an air-liquid interface of the cultivation fluid. In some embodiments, it may be desirable to maintain the cultivation fluid at a pH between 9.5 and 11.5 and supply the cultivation fluid with carbonate and bicarbonate ions.

An output of the algae cultivation unit (1) is an algae slurry. An algae slurry is defined as a mixture of algae suspended in a liquid. In some embodiments the algae form a slurry within an algae growth media. In some cases, the algae growth media is depleted of nutrients (e.g., carbon dioxide) associated with the cultivation of algae within the media. The algae slurry may move through a fluid connection such as a slurry conduit (2) configured to transfer the algae slurry from the algae cultivation unit (1) to the harvest system (3).

Harvest system (3) produces a permeate and a retentate from the algae slurry provided by the slurry conduit (2) between the algae cultivation unit (1) and the harvest system (3). Examples of suitable harvest systems are described in U.S. Pat. Nos. 10,501,721 B2, 10,351,815 B2, and U.S. Patent Publication No. US2020/0299636A1, each of which are incorporated by reference, in their entirety and for all purposes, herein. In some embodiments, referring to the harvest system (3), the retentate may be algae (i.e., an algae product). In some embodiments, the retentate may be a concentrated algae product. In some embodiments, the permeate may be algae growth media. In some cases, the permeate may be recycled. For example, the permeate from a harvest system (3) may be returned by a recycled media stream (4) to the algae cultivation system (1). In some cases the recycled media stream (4) is contacted with a source of carbon dioxide to absorb carbon dioxide prior to returning to the cultivation system (1). The source of carbon dioxide may be in the form of air, i.e., atmosphere. In other cases, the source of carbon dioxide may provide concentrated carbon dioxide, such as when the source is flue gas. In some embodiments, carbonic anhydrase will persist in the algae cultivation media for more than one cycle through the algae cultivation unit (1) and the harvest system (3) such that there is carbonic anhydrase in the recycled media (4) that increases the efficiency of carbon dioxide absorption in the recycled media stream (4). In other cases, the permeate from a harvest system (3) may be optionally supplied to a microorganism cultivation unit (5) via a secondary recycled media conduit (6) configured to transfer at least a portion of the recycled media from the harvest system (3) to the microorganism cultivation unit (5).

Microorganism cultivation unit (5) is configured to produce a microorganism slurry in a growth media under conditions in which one or more microorganisms produces and excretes carbonic anhydrase into the growth media. A microorganism slurry is defined as a mixture of one or more microorganisms suspended in a liquid. In some embodiments the microorganisms form a slurry within a growth media. In some cases, the growth media is replete with nutrients (e.g., carbon dioxide) associated with the cultivation of microorganism within the media. Without wishing to be bound by theory, carbonic anhydrase greatly accelerates the hydration of dissolved CO₂ in solution thereby increasing the concentration of CO₂ in the growth media directly from air (e.g., gases within the atmosphere). In some embodiments, the growth media in the microorganism unit (5) provides a condition in which the microorganism excretes the carbonic anhydrase into the media. In some embodiments the microorganism lyses when mixed with the main recycled media (4) or in the cultivation unit (1). For example, the main recycled media (4) may be at a high enough pH or salinity relative to the pH or salinity in the growth media in the microorganism cultivation unit (5) to lyse the microorganism as it is transferred into the algae cultivation unit (1). In other embodiments, the cultivation fluid in the algae cultivation unit (1) provides a condition in which the microorganism excretes the carbonic anhydrase into the cultivation fluid, or the microorganism lyses in the cultivation fluid after it is transferred to the algae cultivation unit (1).

Microorganisms expressing carbonic anhydrase may include cyanobacteria, bacteria, algae, fungi, or archaebacteria. In some cases, the microorganism may include Chlamydomonas reinhardtii, Methanosarcina thermophila, Porphyridium purpureum, Dunaliella salina, Saccharomyces cerevisiae, Schizosaccharomyces pombe, Nitzschia sp., Nitzschia inconspicua, Escherichia coli, and any combinations thereof. In some embodiments, the microorganism or combination of microorganisms may be genetically engineered to optimally produce carbonic anhydrase, for example as described in Supuran, C., & Capasso, C. (2017). An Overview of Bacterial Carbonic Anhydrase [Review of An Overview of Bacterial Carbonic Anhydrase]. Metabolites, 7(56), 1-19 and Hwang, I. S., Kim, J. H., & Jo, B. H. (2021). Enhanced Production of a Thermostable Carbonic Anhydrase in Escherichia coli by using a Modified NEXT Tag [Review of Enhanced Production of a Thermostable Carbonic Anhydrase in Escherichia coli by using a Modified NEXT Tag]. Molecules, 26, 5830.

The microorganism cultivation unit (5) may cultivate one or more microorganisms under conditions for production and excretion of carbonic anhydrase. It may be appreciated that conditions for expression or excretion of carbonic anhydrase may be dependent upon the microorganism or microorganisms selected, including conditions such as temperature, pH, nutrient availability, salinity, or sunlight duration and intensity. The non-limiting Example provided herein describes growth conditions under which Nitzschia sp. may be grown to express carbonic anhydrase. In some cases, the microorganism may excrete carbonic anhydrase. The carbonic anhydrase may be excreted substantially into the microorganism growth media. In other cases, the microorganism may express carbonic anhydrase within the cell membrane or cell wall.

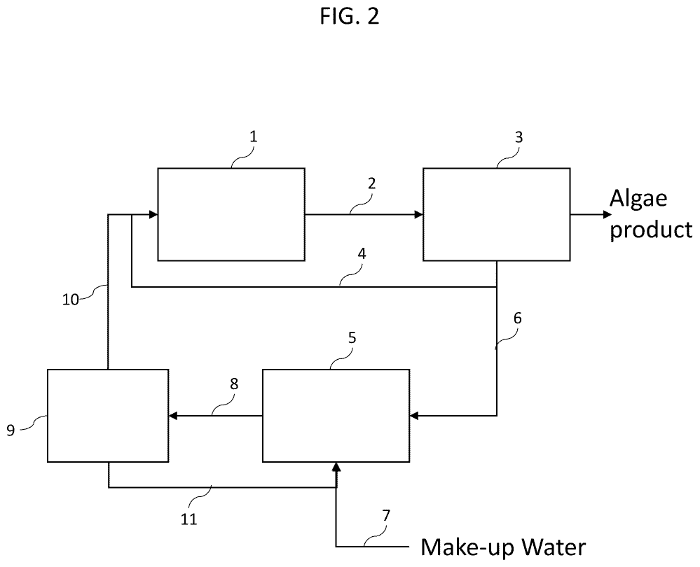

The growth media conduit (8) may transfer at least a portion of the microorganism slurry from the microorganism cultivation unit (5) to the algae cultivation unit (1). In some cases, such as under conditions where carbonic anhydrase may be excreted substantially into the microorganism growth media, it may be desirable to separate the microorganism from the growth media (e.g., by filtration, by centrifugation, by sedimentation, by air floatation, by flocculation, or by combinations thereof), as shown in FIG. 2. Referring now to FIG. 2, the algae cultivation system may further include a microorganism harvesting system (9) configured to separate the microorganism from the growth media, a filtered media conduit (10) configured to transfer at least a portion of the growth media to the algae cultivation unit (1), and a recycled microorganism slurry conduit (11) configured to optionally transfer at least a portion of the recycled microorganism slurry from the harvesting system (9) back to the microorganism cultivation unit (5). Examples of suitable harvest systems are described in U.S. Pat. Nos. 10,501,721 B2, 10,351,815 B2, and U.S. Patent Publication No. US2020/0299636A1, each of which are incorporated by reference, in their entirety and for all purposes, herein.

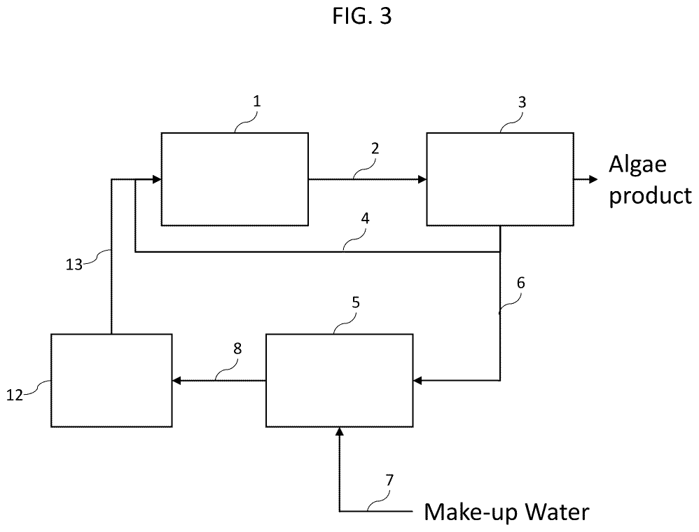

Some microorganisms express carbonic anhydrase within the cell membrane or cell wall. When using such microorganisms in the disclosed system, it may be desirable for the cell wall or cell membrane to be disrupted, thereby exposing carbonic anhydrase to the growth media. Disrupting the microorganism's cell membrane or cell wall may be accomplished by various methods already characterized in the art, including mechanical disruption, liquid homogenization, high frequency sound waves (sonication), freeze/thaw cycles, manual grinding, solvents and/or detergents to solubilize the cell membrane components, enzymes, or osmotic shock. Referring to FIG. 3, an embodiment of the algae cultivation system includes a lysis system (12) configured to disrupt the cell wall and/or cell membrane of the microorganism and a lysed microorganism slurry conduit (13) configured to transfer at least a portion of the lysed microorganism including the growth media and microorganisms produced from the lysis system (12) to the algae cultivation unit (1).

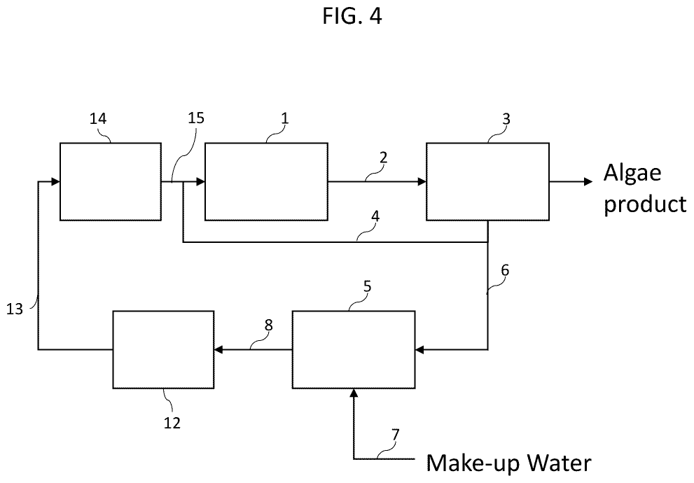

In some cases, it may be desirable to separate the disrupted microorganism from the growth media. Referring to FIG. 4, the algae cultivation system may further include a filtration system (14) configured to separate the disrupted microorganism from the growth media and a second filtered media conduit (15) configured to transfer at least a portion of the growth media, preferably enriched with carbonic anhydrase, from the lysis system (12) to the algae cultivation unit (1). Examples of suitable filtration systems (14) are described in U.S. Pat. Nos. 10,501,721 B2, 10,351,815 B2, and U.S. Patent Publication No. US2020/0299636A1, each of which are incorporated by reference, in their entirety and for all purposes, herein.

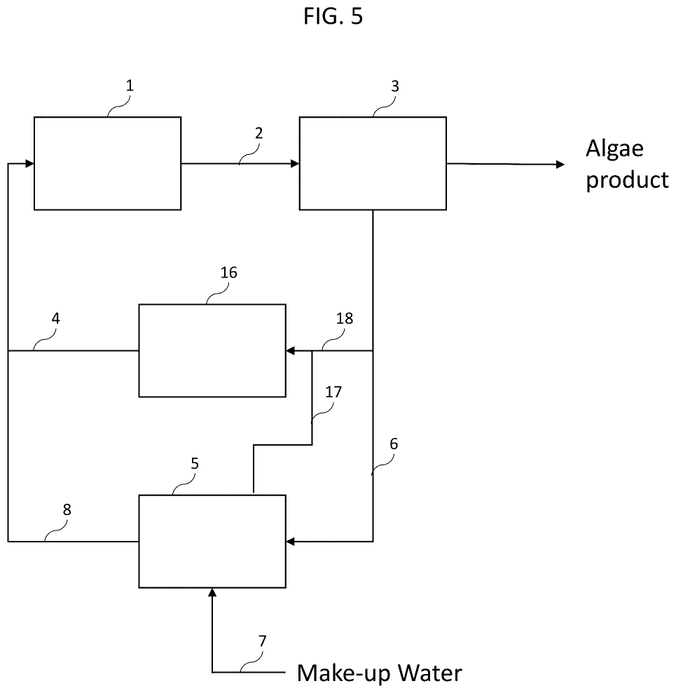

As described in U.S. Pat. Nos. 10,501,721 B2, 10,351,815 B2, and U.S. Patent Publication No. US2020/0299636A1, carbon dioxide may be absorbed from a carbon dioxide containing gas, such as air, into the recycled media. Referring now to FIG. 5, carbon dioxide is absorbed from the air in carbon dioxide addition system (16) which includes a gas-liquid contactor, and in which some of the carbonate ions in the main recycled media (18) are converted to bicarbonate ions. The bicarbonate ion enriched main recycled media (4) is added to the algae cultivation unit (1). Non-limiting examples of gas-liquid contactors include an absorber, a packed bed absorber with structured or unstructured packing, a cross flow packed bed absorber, a mechanical surface aerator, a wave generator, and a two-phase static mixer. In some embodiments the carbon dioxide addition system (16) includes a holding reservoir before and/or after the gas-liquid contactor to balance the flows from harvesting, to and from the gas-liquid contactor, and to cultivation.

In some embodiments the gas-liquid contactor is in the holding reservoir, such as a rotating aeration disc or other mechanical surface aerator. In some embodiments, the gas-liquid contactor in the carbon dioxide addition system (16) contacts the recycled media (4) one or more of air, a moderately concentrated CO₂ source, e.g. 1% to 20% CO₂ by volume, such as combustion flue gas, or a concentrated CO₂ source, e.g. 20% to 100% CO₂ by volume such as fermentation gas or a gas from direct air capture process. In some embodiments, carbonic anhydrase will persist in the algae cultivation media for more than one cycle through the algae cultivation unit (1) and the harvest system (3) such that there is carbonic anhydrase in the recycled media (4) that increases the efficiency of carbon dioxide absorption in the carbon dioxide addition system (16).

In some embodiments at least a portion of the growth media or microorganism slurry is added to the carbon dioxide addition system (16) through the alternate growth media conduit (17) from the microorganism cultivation unit (5) such the main recycled media steam (4) is combined with the alternate growth media (17) in the carbon dioxide addition system (16). In some embodiments the alternate growth media (17) is a microorganism slurry, for example if the carbonic anhydrase is excreted, if the carbonic anhydrase the surface of the microorganism, or if the carbonic anhydrase is inside the microorganism and the microorganism lyses from mixing of the alternate growth media (17) with the main recycled media (4). In some embodiments the microorganisms in the alternate growth media (17) are lysed prior to entering the carbon dioxide addition system (16). In some embodiments the microorganisms or debris from lysed microorganisms are filtered from the alternate growth media (17) prior to entering the carbon dioxide addition system (16).

In may be desirable to further process the carbonic anhydrase (e.g., concentrate, isolate, or purify) in the microorganism growth media before providing the carbonic anhydrase to the direct air capture algae cultivation unit. Therefore, the system may further comprise a processing unit that fluidically connects the microorganism cultivation unit (5) and the algae cultivation unit (1) or the carbon dioxide addition system (16).

In some embodiments, a carbon dioxide absorber may be incorporated into the algae cultivation system. In some embodiments, the absorber may be positioned to receive one or more carbonic anhydrous enriched outputs of the microorganism cultivation unit (5) and/or the harvest system (3) and/or the filtration system (14). Without wishing to be bound by theory, the carbonic anhydrous enriched outputs of the microorganism cultivation unit (5) and/or the harvest system (3) and/or the filtration system (14) may contact a gas containing carbon dioxide in the absorber, where the carbonate ions in the media is converted to bicarbonate ions resulting in a carbonated media. In some embodiments, the absorber may be a packed bed absorber with structured or random packing, a rotating disc gas-liquid contactor, or a two-phase static mixer.

### Methods

Disclosed herein are methods of cultivating an algae culture in a cultivation fluid. The methods may be performed using any of the systems described herein.

In an embodiment, the method comprises:
(i) cultivating a microorganism that produces carbonic anhydrase in a growth media under conditions in which the microorganism produces and excretes the carbonic anhydrase into the growth media; and
(ii) providing the growth media to the algae culture.

In another embodiment, the method comprises:

(i) cultivating a microorganism that produces carbonic anhydrase in a growth media under conditions in which the microorganism produces the carbonic anhydrase;
(ii) lysing the microorganism in the growth media; and (iii) providing the growth media to the algae culture.

In another embodiment, the method comprises:
(i) cultivating a microorganism that produces carbonic anhydrase in a growth media under conditions in which the microorganism produces the carbonic anhydrase;
(ii) providing the growth media to the algae culture;
(iii) harvesting the algae from the algae culture to make an algae slurry product and a recycled cultivation fluid that contains dissolved carbonic anhydrase;
(iv) contacting the cultivation fluid with air or another carbon dioxide containing gas to enrich the bicarbonate ion concentration; and
(v) providing the bicarbonate enriched recycled cultivation fluid to the algae culture.

In another embodiment, the method comprises:
(i) cultivating a microorganism that produces carbonic anhydrase in a growth media under conditions in which the microorganism produces the carbonic anhydrase;
(ii) lysing the microorganism in the growth media;
(iii) providing the growth media to the algae culture;
(iv) harvesting the algae from the algae culture to make an algae slurry product and a recycled cultivation fluid that contains dissolved carbonic anhydrase;
(v) contacting the cultivation fluid with air or another carbon dioxide containing gas to enrich the bicarbonate ion concentration; and
(vi) providing the bicarbonate enriched recycled cultivation fluid to the algae culture.

The terms “lyse” and “lysing” as used herein refer to disrupting the cell wall and/or cell membrane of the microorganism. Disrupting the microorganism's cell membrane or cell wall may be accomplished by various methods already characterized in the art, including mechanical disruption, liquid homogenization, high frequency sound waves (sonication), freeze/thaw cycles, manual grinding, solvents and/or detergents to solubilize the cell membrane components, enzymes, or osmotic shock.

In some embodiments, the method includes growing the algae culture in a cultivation system configured to capture carbon dioxide directly from air. As described previously, examples of direct air capture algae cultivation systems and methods are described in U.S. Pat. Nos. 10,501,721 B2, 10,351,815 B2, U.S. Patent Publication No. US2020/0299636A1, and U.S. Patent Publication No. US2021/0386029A1 each of which are incorporated by reference, in their entireties and for all purposes, herein. For example, one method of capturing carbon dioxide from air may include applying bore waves through the cultivation fluid at a bore wave frequency sufficient to disrupt an air-liquid interface of the cultivation fluid. For example, another method of capturing carbon dioxide from air may include a rotating aeration disc or other mechanical surface aerator to mix air with the cultivation fluid. In some embodiments, it may be desirable to maintain the cultivation fluid at a pH between 9.5 and 11.5 and supply the cultivation fluid with carbonate and bicarbonate ions.

In another embodiment, the method comprises:
(i) cultivating a microorganism that produces carbonic anhydrase in a growth media under conditions in which the microorganism produces the carbonic anhydrase;
(ii) providing the at least a portion growth media to a carbon dioxide addition system wherein carbon dioxide is absorbed in an aqueous media to convert carbonate ions to bicarbonate ions to produce a bicarbonate enriched media;
(iii) providing the bicarbonate enriched media to the algae culture;
(iv) harvesting the algae from the algae culture to make an algae slurry product and a recycled cultivation fluid; and
(v) providing at least a portion of the recycled cultivation fluid to the carbon dioxide addition system where it is mixed with the growth media to facilitate absorption of carbon dioxide.

In another embodiment, the method comprises:
(i) cultivating a microorganism that produces carbonic anhydrase in a growth media under conditions in which the microorganism produces the carbonic anhydrase;
(ii) lysing the microorganism in the growth media;
(iii) providing the at least a portion growth media to a carbon dioxide addition system wherein carbon dioxide is absorbed in an aqueous media to convert carbonate ions to bicarbonate ions to produce a bicarbonate enriched media;
(iv) providing the bicarbonate enriched media to the algae culture;
(v) harvesting the algae from the algae culture to make an algae slurry product and a recycled cultivation fluid; and
(vi) providing at least a portion of the recycled cultivation fluid to the carbon dioxide addition system where it is mixed with the growth media to facilitate absorption of carbon dioxide.

In some embodiments, the method includes growing the algae culture in a cultivation system configured to capture carbon dioxide directly from air. As described previously, Examples of direct air capture algae cultivation systems and methods are described in U.S. Pat. Nos. 10,501,721 B2, 10,351,815 B2, U.S. Patent Publication No. US2020/0299636A1, and U.S. Patent Publication No. US2021/0386029A1 each of which are incorporated by reference, in their entireties and for all purposes, herein. For example, one method of capturing carbon dioxide from air may include applying bore waves through the cultivation fluid at a bore wave frequency sufficient to disrupt an air-liquid interface of the cultivation fluid. For example, another method of capturing carbon dioxide from air may include a rotating aeration disc or other mechanical surface aerator to mix air with the cultivation fluid. In some embodiments, it may be desirable to maintain the cultivation fluid at a pH between 9.5 and 11.5 and supply the cultivation fluid with carbonate and bicarbonate ions.

In some embodiments the method includes capturing carbon dioxide from air directly into the recycled cultivation fluid. A non-limiting example of direct air capture into a media containing carbonate ions includes a packed bed absorber as described in U.S. Pat. Nos. 10,501,721 B2, 10,351,815 B2, 9,095,813, and U.S. Patent Publication No. US2020/0299636A1.

In some embodiments, the growth media contains the microorganism in step (ii). In other embodiments, the microorganism may be removed from the growth media before step (ii). As described previously, microorganisms expressing carbonic anhydrase may include cyanobacteria, bacteria, algae, fungi, or archaebacteria. In some cases, the microorganism may include Chlamydomonas reinhardtii, Methanosarcina thermophila, Porphyridium purpureum, Dunaliella salina, Saccharomyces cerevisiae, Schizosaccharomyces pombe, Nitzschia spp. (such as Nitzschia inconspicua, Nitzschia acicularis, Nitzschia amphibia, Nitzschia angustata, Nitzschia brevissima, Nitzschia clausii, Nitzschia denticule, Nitzschia disputata, Nitzschia dissipata, Nitzschia filiformis, Nitzschia fonticuli, Nitzschia frigida, Nitzschia gracilis, Nitzschia frigida, Nitzschia heuflerania, Nitzschia frigida, Nitzschia lacuum, Nitzschia palea, Nitzschia perminuta, Nitzschia pusill, Nitzschia recta, Nitzschia sigma, Nitzschia sigmoidea, Nitzschia sinuate, Nitzschia tubicola), Escherichia coli, and any combinations thereof. In some embodiments, the microorganism or combination of microorganisms may be genetically engineered to optimally produce carbonic anhydrase. Without wishing to be bound by theory, carbonic anhydrase greatly accelerates the hydration of dissolved CO₂ in solution thereby increasing the concentration of CO₂ in the growth media directly from air (e.g., gases within the atmosphere).

In any of the previously described systems and methods, the microorganism may be the same genus and species as the algae cultivated in the algae cultivation unit. In one embodiment, the microorganism may be Nitzschia inconspicua, cultivated under conditions where it produces carbonic anhydrase, and the algae cultivated in the algae cultivation unit is also Nitzschia inconspicua. This enables adding carbonic anhydrase without adding microorganisms of different genus and species to the algae cultivation unit and without purification.

In an embodiment of any of the previously describes systems and methods, the carbonic anhydrase produced by the microorganism cultivated in the microorganism cultivation unit is supplied to the algae cultivation unit for utilization by the algae present in the algae cultivation unit. In some instances, both the microorganism cultivation unit and the algae cultivation unit contain the same microorganism (e.g., Nitzschia inconspicua). In some instances, the microorganism cultivation unit and the algae cultivation unit may be operated under different growing conditions. For example, the microorganism cultivation unit may be operated under conditions where the microorganism produces relatively more carbonic anhydrase than the algae present in the algae cultivation unit. In other instances, both the microorganism cultivation unit and the algae cultivation unit may be operated under the same or substantially the same growing conditions. In some cases, the microorganism in the microorganism cultivation unit may produce relatively the same amount of carbonic anhydrase as the algae present in the algae cultivation unit. It may be desirable to make the carbonic anhydrase accessible for utilization by the algae by lysing the microorganism.

### EXAMPLE CLAUSES

### Example Clause 1

An algae cultivation system comprising:
an algae cultivation unit (1) configured to produce an algae slurry in a cultivation fluid;
   a microorganism cultivation unit (5) configured to produce a microorganism slurry in a growth media under conditions in which one or more microorganisms produce carbonic anhydrase; and
   a growth media conduit (8) configured to transfer at least a portion of the microorganism slurry from the microorganism cultivation unit (5) to the algae cultivation unit (1).

### Example Clause 2

The algae cultivation system of clause 1, wherein the one or more microorganisms produce and excrete carbonic anhydrase into the growth media.

### Example Clause 3

An algae cultivation system comprising:
an algae cultivation unit (1) configured to produce an algae slurry in a cultivation fluid;
a microorganism cultivation unit (5) configured to produce a microorganism slurry in a growth media under conditions in which one or more microorganisms produces and excretes carbonic anhydrase into the growth media;
   a microorganism harvesting system (9) configured to separate the microorganism from the growth media and generate a filtered media;
   a growth media conduit (8) configured to transfer the microorganism slurry from the microorganism cultivation unit (5) to the microorganism harvesting system (9); and
   a filtered media conduit (10) configured to transfer at least a portion of the filtered media from the microorganism harvesting system (9) to the algae cultivation unit (1).

### Example Clause 4

An algae cultivation system comprising:
an algae cultivation unit (1) configured to produce an algae slurry in a cultivation fluid;
   a microorganism cultivation unit (5) configured to produce a microorganism slurry in a growth media under conditions in which one or more microorganisms produces carbonic anhydrase;
   a lysis system (12) configured to lyse the microorganism and release carbonic anhydrase into the growth media;
   a growth media conduit (8) configured to transfer the microorganism slurry from the microorganism cultivation unit (5) to the lysis system (12);
   a lysed microorganism slurry conduit (13) configured to transfer at least a portion of the lysed microorganism from the lysis system (12) to the algae cultivation unit (1).

### Example Clause 5

An algae cultivation system comprising:
an algae cultivation unit (1) configured to produce an algae slurry in a cultivation fluid;
   a harvest system (3) configured to produce a concentrated algae product from the algae slurry;
   a slurry conduit (2) configured to transfer the algae slurry from the algae cultivation unit (1) to the harvest system (3);
   a microorganism cultivation unit (5) configured to produce a microorganism slurry in a growth media under conditions in which one or more microorganisms produces carbonic anhydrase;
   a lysis system (12) configured to lyse the microorganism and release carbonic anhydrase into the growth media;
   a growth media conduit (8) configured to transfer the microorganism slurry from the microorganism cultivation unit (5) to the lysis system (12);
   a filtration system (14) configured to separate the microorganism suspended cell debris from the growth media and produce a filtered lysis media;
   a lysed microorganism slurry conduit (13) configured to transfer at least a portion of the lysed microorganism slurry from the lysis system (12) to the filtration system (14); and
   a filtered lysis media conduit (15) configured to transfer at least a portion of the filtered lysis media from the filtration system (14) to the algae cultivation unit (1).

### Example Clause 6

The algae cultivation system of any one of the preceding clauses further comprising:

a harvest system (3) configured to produce a concentrated algae product from the algae slurry and a recycled cultivation fluid;
a slurry conduit (2) configured to transfer the algae slurry from the algae cultivation unit (1) to the harvest system (3); and
a first recycled cultivation fluid conduit (4) configured to supply the recycled cultivation fluid to algae cultivation unit (1).

### Example Clause 7

The algae cultivation system of algae cultivation system of clause 6 further comprising:

a holding reservoir (16)
a second recycle fluid conduit (18) configured to supply a recycled cultivation fluid from the harvest system (3) to the holding reservoir (16);
and wherein said first recycle fluid conduit fluidly connects the holding reservoir to the algae cultivation unit (1).

### Example Clause 8

The algae cultivation system of clause 7, wherein the holding reservoir is connected to or contains a gas-liquid contactor that contacts the recycled cultivation fluid with air.

### Example Clause 9

The algae cultivation system of clause 8, wherein the gas-liquid contactor is selected from: an absorber with structured packing, an absorber with random packing, a crossflow absorber with structured packing, a cross flow absorber with random packing, a rotating aeration disc, a mechanical surface aerator, a wave generator, an air bubble generator, and any combinations thereof.

### Example Clause 10

The algae cultivation system of any one of the preceding clauses, further comprising:
a secondary recycled media conduit (6) configured to transfer at least a portion of the cultivation fluid from the harvest system (3) to the microorganism cultivation unit (5).

### Example Clause 11

The algae cultivation system of any one of the preceding clauses, further comprising:
a make-up water conduit (7) configured to transfer water to the microorganism cultivation unit (5).

### Example Clause 12

The algae cultivation system of any one of the preceding clauses, wherein the algae cultivation system is configured to capture carbon dioxide directly from air.

### Example Clause 13

The algae cultivation system of any one of clauses 8-11, wherein the system is configured to transfer at least a portion of the microorganism slurry, filtered media, lysed microorganism slurry, or a filtered microorganism slurry to the recycle cultivation fluid before the fluid enters the gas-liquid contactor.

### Example Clause 14

The system of any one of the preceding clauses, wherein the microorganism is selected from an algae, a bacteria, or a fungi.

### Example Clause 15

The system of clause 14, wherein the microorganism is from one or more genera selected from Chlamydomonas, Methanosarcina, Porphyridium, Dunaliella, Saccharomyces, Schizosaccharomyces, Nitzschia, Escherichia, Coccomyxa, and any combinations thereof.

### Example Clause 16

The system of clause 14, wherein the microorganism is selected from Chlamydomonas reinhardtii, Methanosarcina thermophila, Porphyridium purpureum, Dunaliella salina, Saccharomyces cerevisiae, Schizosaccharomyces pombe, Nitzschia inconspicua, Nitzschia acicularis, Nitzschia amphibia, Nitzschia angustata, Nitzschia brevissima, Nitzschia clausii, Nitzschia denticule, Nitzschia disputata, Nitzschia dissipata, Nitzschia filiformis, Nitzschia fonticuli, Nitzschia frigida, Nitzschia gracilis, Nitzschia frigida, Nitzschia heuflerania, Nitzschia frigida, Nitzschia lacuum, Nitzschia palea, Nitzschia perminuta, Nitzschia pusill, Nitzschia recta, Nitzschia sigma, Nitzschia sigmoidea, Nitzschia sinuate, Nitzschia tubicola, Escherichia coli, Coccomyxa subellipsoides, and any combinations thereof.

### Example Clause 17

The system of clause 14, wherein the microorganism is genetically engineered to produce or excrete carbonic anhydrase.

### Example Clause 18

A method of cultivating an algae culture using the system of any one of clauses 1-17.

### Example Clause 19

A method of cultivating an algae culture in a cultivation fluid comprising:
(i) cultivating a microorganism that produces carbonic anhydrase in a growth media under conditions in which the microorganism produces and excretes carbonic anhydrase into the growth media; and
(ii) providing the at least a portion of growth media to the algae culture.

### Example Clause 20

The method of clause 18 wherein the algae culture is grown in a cultivation system configured to capture carbon dioxide directly from air.

### Example Clause 21

The method of clause 19 or clause 20, wherein the growth media contains the microorganism in step (ii).

### Example Clause 22

The method of clause 19 or clause 20, further comprising removing the microorganism from the growth media before step (ii).

### Example Clause 23

The method of any of clauses 19-22, wherein the microorganism is selected from an algae, a bacteria, or a fungi.

### Example Clause 24

The method of clause 19, wherein the microorganism is from one or more genera selected from Chlamydomonas, Methanosarcina, Porphyridium, Dunaliella, Saccharomyces, Schizosaccharomyces, Nitzschia, Escherichia, Coccomyxa, and any combinations thereof.

### Example Clause 25

The method of clause 19, wherein the microorganism is selected from Chlamydomonas reinhardtii, Methanosarcina thermophila, Porphyridium purpureum, Dunaliella salina, Saccharomyces cerevisiae, Schizosaccharomyces pombe, Nitzschia inconspicua, Nitzschia acicularis, Nitzschia amphibia, Nitzschia angustata, Nitzschia brevissima, Nitzschia clausii, Nitzschia denticule, Nitzschia disputata, Nitzschia dissipata, Nitzschia filiformis, Nitzschia fonticuli, Nitzschia frigida, Nitzschia gracilis, Nitzschia frigida, Nitzschia heuflerania, Nitzschia frigida, Nitzschia lacuum, Nitzschia palea, Nitzschia perminuta, Nitzschia pusill, Nitzschia recta, Nitzschia sigma, Nitzschia sigmoidea, Nitzschia sinuate, Nitzschia tubicola, Escherichia coli, Coccomyxa subellipsoides, and any combinations thereof.

### Example Clause 26

The method of clause 19, wherein the microorganism is genetically engineered to produce or excrete carbonic anhydrase.

### Example Clause 27

A method of cultivating an algae culture in a cultivation fluid comprising:
(i) cultivating a microorganism that produces carbonic anhydrase in a growth media under conditions in which the microorganism produces the carbonic anhydrase;
(ii) lysing the microorganism in the growth media; and
(iii) providing at least a portion of the growth media to the algae culture.

### Example Clause 28

The method of clause 27, wherein the algae culture is grown in a cultivation system configured to capture carbon dioxide directly from air.

### Example Clause 29

The method of clause 27-28, wherein the growth media contains the suspended microorganism debris in step (iii).

### Example Clause 30

The method of any one of clauses 27-29, further comprising removing the suspended microorganism from the growth media before step (iii).

### Example Clause 31

The method of any of clauses 27-30, wherein the microorganism is selected from an algae, a bacteria, or a fungi.

### Example Clause 32

The method of clause 27, wherein the microorganism is from one or more genera selected from Chlamydomonas, Methanosarcina, Porphyridium, Dunaliella, Saccharomyces, Schizosaccharomyces, Nitzschia, Escherichia, Coccomyxa, and any combinations thereof.

### Example Clause 33

The method of clause 27, wherein the microorganism is selected from Chlamydomonas reinhardtii, Methanosarcina thermophila, Porphyridium purpureum, Dunaliella salina, Saccharomyces cerevisiae, Schizosaccharomyces pombe, Nitzschia inconspicua, Nitzschia acicularis, Nitzschia amphibia, Nitzschia angustata, Nitzschia brevissima, Nitzschia clausii, Nitzschia denticule, Nitzschia disputata, Nitzschia dissipata, Nitzschia filiformis, Nitzschia fonticuli, Nitzschia frigida, Nitzschia gracilis, Nitzschia frigida, Nitzschia heuflerania, Nitzschia frigida, Nitzschia lacuum, Nitzschia palea, Nitzschia perminuta, Nitzschia pusill, Nitzschia recta, Nitzschia sigma, Nitzschia sigmoidea, Nitzschia sinuate, Nitzschia tubicola, Escherichia coli, Coccomyxa subellipsoides, and any combinations thereof.

### Example Clause 34

The method of clause 27, wherein the microorganism is genetically engineered to produce or excrete carbonic anhydrase.

### Example Clause 35

The method of any of clauses 29 to 31, wherein at least a portion of the algae culture is cultivated in a cultivation media, and at least a portion of the algae culture is harvested to produce a more concentrated algae product slurry and a recycled cultivation fluid that is provided to the algae culture.

### Example Clause 36

The method of clause 35, wherein at least a portion of the recycled cultivation fluid is used as part of the growth media for cultivating the microorganism.

### Example Clause 37

The method of clauses 35 or 36, wherein prior to providing the growth media to the cultivation system:
(i) the growth media is mixed with at least a portion of the recycled cultivation fluid; and
(ii) the growth media and at least a portion of the recycled cultivation fluid is contacted with air in a gas-liquid contactor.

### Example Clause 38

The method of clause 37, wherein the gas-liquid contactor is selected from: an absorber with structured packing, an absorber with random packing, a crossflow absorber with structured packing, a cross flow absorber with random packing, a rotating aeration disc, a mechanical surface aerator, a wave generator, an air bubble generator, and any combinations thereof.

### Example Clause 39

The methods and systems of any of the preceding clauses, wherein the microorganism is the same genus and species as an alga in the algae cultivation unit configured to produce an algae slurry in a cultivation fluid.

### Example Clause 40

The methods and systems of clause 39, wherein the microorganism is Nitzschia insconspicua and the algae is Nitzschia inconspicua.

### EXAMPLES

The following examples are presented by way of illustration and are not meant to be limiting in any way.

### Assessment of Carbonic Anhydrase Activity in Algae Cultures

### Reagents

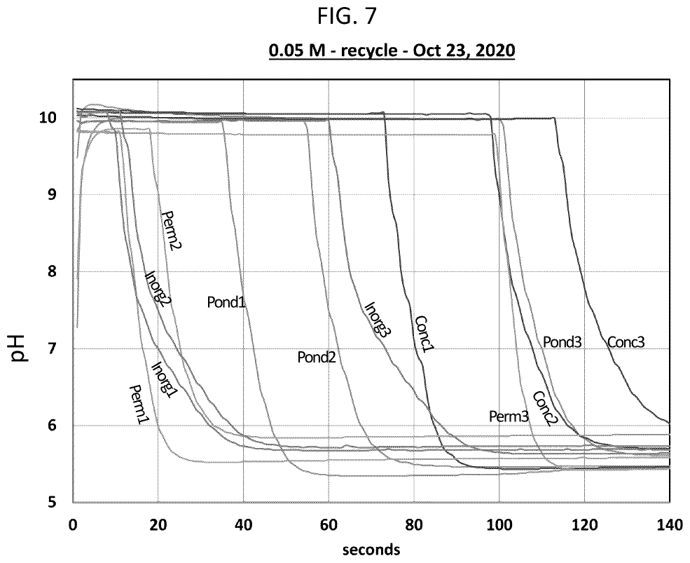

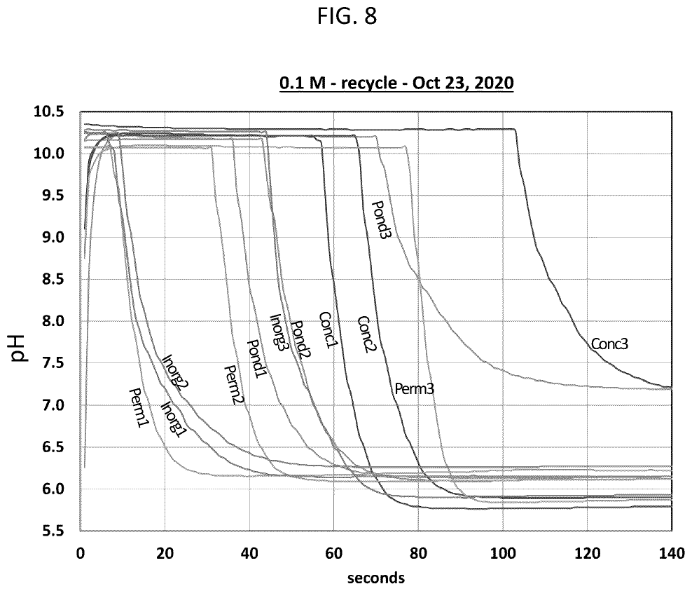

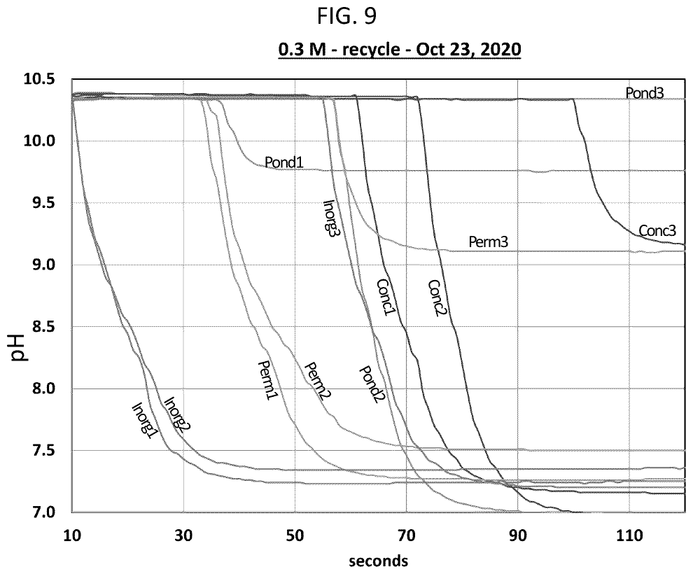

Samples were collected in triplicate from five different algae cultivation ponds growing Nitzschia sp, each pond having a different molarity of Na⁺. See Table 1 and FIGS. 5-9. The samples were collected and processed using an algae harvester.

Each sample was processed into four subsamples:
(a) direct sample from algae pond (green lines)
(b) the concentrated algae sample from the algae harvester (purple lines)
(c) the permeate from the algae harvester (blue lines)
(d) inorganic media at matching conditions, pH, and temperature (red lines).

Carbon dioxide saturated deionized water was generated by adding at least 250 mL of deionized water to a container. The container was sparged with carbon dioxide for at least 20 minutes, achieving a pH of about 4.5. The headspace of the container was additionally filled with carbon dioxide.

### Test Procedure

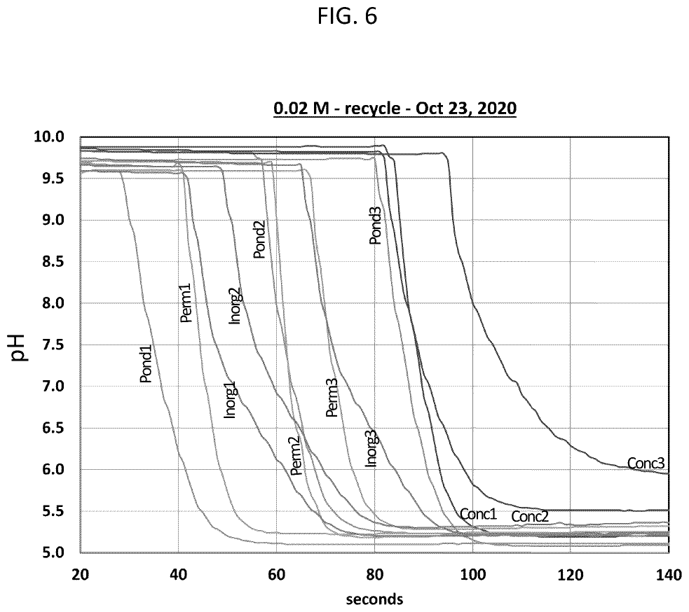

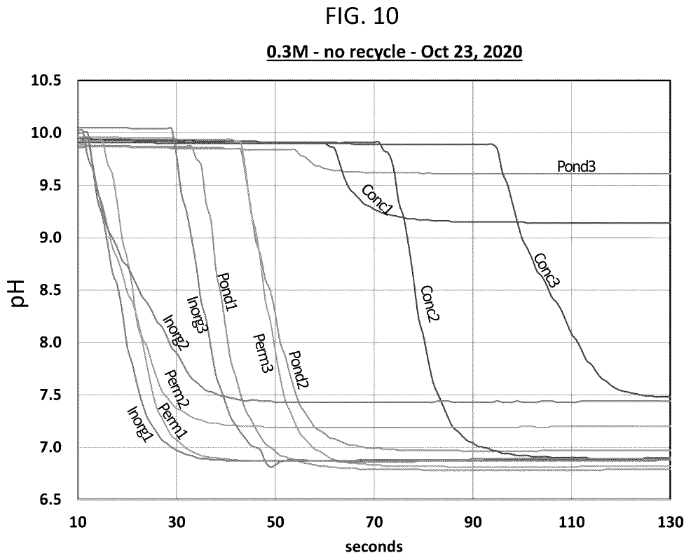

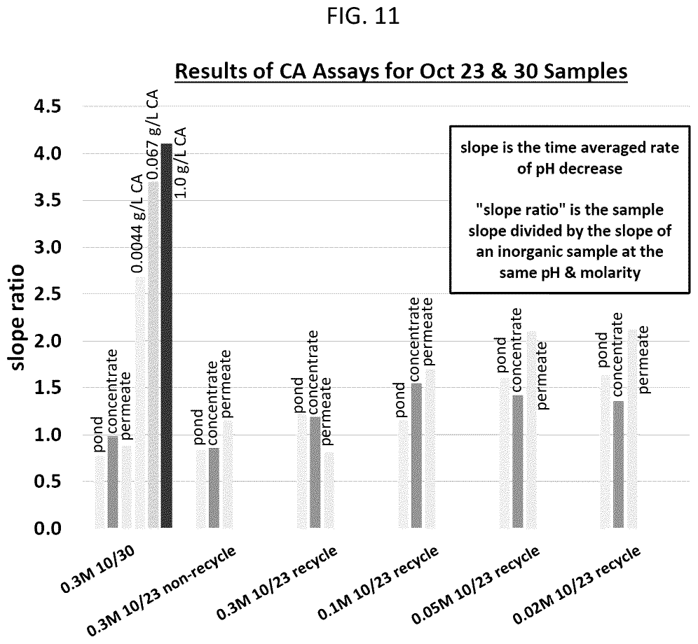

The deionized water (140 mL) and one of subsamples (a)-(d) (10 mL) were added to a beaker. The beaker was mixed at a constant speed using a stir bar. A YSI probe was added (without cover) and set for a 1-sec sampling interval. CO₂ sparge was turned off and 50 mL of CO₂-saturated water was transferred to the beaker. The data (Table 1) was plotted (pH vs time) (FIGS. 6-10). The slopes and three CA ratios from the plots were calculated and are presented in FIG. 11. In addition, specific amounts of carbonic anhydrase were added to a 10/30 sample from a 0.3M pond (‘0.3M 10/30’ in FIG. 11).

### Results

TABLE 1
pH of samples

| | |
|---|---|
| Date | Oct. 23, 2020 |

| | | | | Slurry | Recycle | | |
|---|---|---|---|---|---|---|---|
| | Test, | Re- | Harvest | Conc | Media | pH | pH |
| Pond | M Na | cycle | Vol, L | ratio | pH | pond | permeate |
| 69 | 0.02 | Y | 80 | 20 | 10.44 | 10.53 | 10.47 |
| 70 | 0.05 | Y | 74 | 18.5 | 10.57 | 10.62 | 10.62 |
| 71 | 0.1 | Y | 74 | 18.5 | 10.27 | 10.60 | 10.63 |
| 72 | 0.3 | Y | 70 | 17.5 | 9.70 | 10.12 | 10.26 |
| 73 | 0.3 | N | 30 | 7.5 | 8.25 | 9.6 | 9.8 |

| A | B | C | D |
|---|---|---|---|
| Direct | Zobi | Zobi | Inorganic matching |
| from | concentrate | permeate | conductivity |
| pond | | | and pH, temp |

The permeate with no algae cells appear, in the three lower molarity cases, to have the fastest overall pH change. The algae concentrate from algae harvest system had somewhat slower rates of pH change. This could mean that more carbonic anhydrase is in solution than on the cell surface. Based on the added carbonic anhydrase dosages in the ‘0.3M 10/30’ sample in FIG. 11, the total improvement in carbon dioxide absorption rate of a little over two times the non-CA baseline for the 0.02M and 0.05M ponds, is similar to what would be expected from adding approximately 0.0036 g/liter of carbonic anhydrase. There are significantly diminishing returns from going to much higher concertation of added carbonic anhydrase. Adding 15 times more carbonic anhydrase than 0.0044 increased the absorption rate by about 40%, and adding 227 times more carbonic anhydrase increased the absorption rate by about 60%.

The invention has been described according to one or more preferred embodiments, and it should be appreciated that many equivalents, alternatives, variations, and modifications, aside from those expressly stated, are possible and within the scope of the invention.

The preceding discussion is presented to enable a person skilled in the art to make and use embodiments of the invention. Various modifications to the illustrated embodiments will be readily apparent to those skilled in the art, and the generic principles herein can be applied to other embodiments and applications without departing from embodiments of the invention. Thus, embodiments of the invention are not intended to be limited to embodiments shown, but are to be accorded the widest scope consistent with the principles and features disclosed herein. The detailed description is to be read with reference to the figures, in which like elements in different figures have like reference numerals. The figures, which are not necessarily to scale, depict selected embodiments and are not intended to limit the scope of embodiments of the invention. Skilled artisans will recognize the examples provided herein have many useful alternatives and fall within the scope of embodiments of the invention.

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

Unless specified or limited otherwise, the terms “mounted,” “connected,” “supported,” and “coupled” and variations thereof are used broadly and encompass both direct and indirect mountings, connections, supports, and couplings. Further, “connected” and “coupled” are not restricted to physical or mechanical connections or couplings.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

We claim:

1. An algae cultivation system comprising:
an algae cultivation unit configured to produce an algae slurry in a cultivation fluid;
a microorganism cultivation unit configured to produce a microorganism slurry in a growth media under conditions in which one or more microorganisms produce carbonic anhydrase; and
a growth media conduit configured to transfer at least a portion of the microorganism slurry from the microorganism cultivation unit to the algae cultivation unit.

2. The algae cultivation system of claim 1, wherein the one or more microorganisms produce and excrete carbonic anhydrase into the growth media.

3. The algae cultivation system of claim 1, further comprising:
a harvest system configured to produce a concentrated algae product from the algae slurry and a recycled cultivation fluid;
a slurry conduit configured to transfer the algae slurry from the algae cultivation unit to the harvest system; and
a first recycled cultivation fluid conduit configured to supply the recycled cultivation fluid to the algae cultivation unit.

4. The algae cultivation system of claim 3, further comprising:
a holding reservoir
a second recycled cultivation fluid conduit configured to supply a recycled cultivation fluid from the harvest system to the holding reservoir;
and wherein the first recycled cultivation fluid conduit fluidly connects the holding reservoir to the algae cultivation unit.

5. The algae cultivation system of claim 4, wherein the holding reservoir is connected to or contains a gas-liquid contactor that contacts the recycled cultivation fluid with air.

6. The algae cultivation system of claim 3, further comprising:
a secondary recycled media conduit configured to transfer at least a portion of the cultivation fluid from the harvest system to the microorganism cultivation unit.

7. The algae cultivation system of claim 1, further comprising:
a make-up water conduit configured to transfer water to the microorganism cultivation unit.

8. The algae cultivation system of claim 1, wherein the algae cultivation system is configured to capture carbon dioxide directly from air.

9. The algae cultivation system of claim 5, wherein the system is configured to transfer at least a portion of the microorganism slurry, filtered media, lysed microorganism slurry, or a filtered microorganism slurry to the recycle cultivation fluid before the fluid enters the gas-liquid contactor.

10. The algae cultivation system of claim 1, wherein the microorganism is one of an algae, a bacteria, or a fungi.

11. The algae cultivation system of claim 10, wherein the microorganism is from one or more genera selected from Chlamydomonas, Methanosarcina, Porphyridium, Dunaliella, Saccharomyces, Schizosaccharomyces, Nitzschia, Escherichia, Coccomyxa, and any combinations thereof.

12. An algae cultivation system comprising:
an algae cultivation unit configured to produce an algae slurry in a cultivation fluid;
a microorganism cultivation unit configured to produce a microorganism slurry in a growth media under conditions in which one or more microorganisms produces carbonic anhydrase;
a lysis system configured to lyse the microorganism and release carbonic anhydrase into the growth media;
a growth media conduit configured to transfer the microorganism slurry from the microorganism cultivation unit to the lysis system;
a lysed microorganism slurry conduit configured to transfer at least a portion of the lysed microorganism from the lysis system to the algae cultivation unit.

13. The algae cultivation system of claim 12, further comprising:
a harvest system configured to produce a concentrated algae product from the algae slurry and a recycled cultivation fluid;
a slurry conduit configured to transfer the algae slurry from the algae cultivation unit to the harvest system; and
a first recycled cultivation fluid conduit configured to supply the recycled cultivation fluid to the algae cultivation unit.

14. The algae cultivation system of claim 13, further comprising:
a holding reservoir; and
a second recycle fluid conduit configured to supply a recycled cultivation fluid from the harvest system to the holding reservoir,
wherein the first recycled cultivation fluid conduit fluidly connects the holding reservoir to the algae cultivation unit.

15. A method of cultivating an algae culture in a cultivation fluid comprising:
(i) cultivating a microorganism that produces carbonic anhydrase in a growth media under conditions in which the microorganism produces and excretes carbonic anhydrase into the growth media; and
(ii) providing the at least a portion of growth media to the algae culture.

16. The method of claim 15, wherein the algae culture is grown in a cultivation system configured to capture carbon dioxide directly from air.

17. The method of claim 15, wherein the growth media contains the microorganism in step (ii).

18. The method of claim 15, further comprising removing the microorganism from the growth media before step (ii).

19. The method of claim 15, wherein the microorganism is one of an algae, a bacteria, or a fungi.

20. The method of claim 15, wherein the microorganism is from one or more genera selected from Chlamydomonas, Methanosarcina, Porphyridium, Dunaliella, Saccharomyces, Schizosaccharomyces, Nitzschia, Escherichia, Coccomyxa, and any combinations thereof.
